(19) United States
(12) Patent Application Publication
(10) Pub. No.: US 2008/0006271 A1
(43) Pub. Date: Jan. 10, 2008

(54) METHOD AND SYSTEM OF GENERATING INDICIA REPRESENTATIVE OF START OF AN INHALATION

(75) Inventors: ALONZO C. AYLSWORTH, WILDWOOD, MO (US); LAWRENCE C. SPECTOR, AUSTIN, TX (US)

Correspondence Address: CONLEY ROSE, P.C.;David A. Rose, P. O. BOX 3267, HOUSTON, TX, 77253-3267, US

(73) Assignee: ACOBA, LLC, CHESTERFIELD, MO (US)

(21) Appl. No.: 11/456,192

(22) Filed: Jul. 8, 2006

(51) Int. Cl. A61M 16/00

(52) U.S. Cl. 128/204.23; 128/204.21

(57) ABSTRACT

A method and system of generating indicia indicative of start of an inhalation. At least some of the illustrative embodiments are methods comprising sensing respirations of a patient, determining the start of an inhalation portion of a respiration, and providing a first indicia indicative of the start of the inhalation portion (the first indicia associated with a representation of the respiration).

### BACKGROUND

Sleep disordered breathing may be diagnosed by having a patient sleep overnight in a sleep lab. The patient is coupled to various sensors (e.g., sensors to monitor respiration, brain waves, respiratory effort belts, and the like), and the patient sleeps while data is collected. A polysomnographer “scores” the data collected to make a determination of whether the patient experienced sleep disordered breathing, such as apnea or hypopnea.

However, in the scoring process it is difficult to correlate in time various events. Consider, for example, a video display device plotting brain waves of the patient during sleep and a corresponding plot of pressure as a function of time from a pressure transducer measuring pressure associated with the patient's respirations, but the pressure is plotted without a horizontal line indicating zero gauge pressure. It is difficult, from merely observing the pressure waveform, to determine a precise point in time when an inhalation started, for example, in relation to a particular brain wave deflection. In a particular example of brain wave deflection indicative of a brain arousal (arousal of the patient from sleep) and respiration illustrated by measured pressure, it is difficult if not impossible to determine if a “break through” breath after an apnea and/or hypopnea caused a brain arousal, or if the brain arousal occurred prior to inhalation. Other parameters of interest may also suffer from the inability to determine with precision the point in time when inhalations and exhalations begin, such as the inhalation time to exhalation time ratio.

### SUMMARY

The problems noted above are solved in large part by a method and system of generating indicia indicative of start of an inhalation. At least some of the illustrative embodiments are methods comprising sensing respirations of a patient determining the start of an inhalation portion of a respiration, and providing a first indicia indicative of the start of the inhalation portion (the first indicia associated with a representation of the respiration).

The disclosed devices and methods comprise a combination of features and advantages which enable them to overcome the deficiencies of the prior art devices. The various characteristics described above, as well as other features, will be readily apparent to those skilled in the art upon reading the following detailed description, and by referring to the accompanying drawings.

### BRIEF DESCRIPTION OF THE DRAWINGS

For a detailed description of the preferred embodiments of the invention, reference will now be made to the accompanying drawings in which:

FIG. 1 shows a sleep study device in accordance with some embodiments;

FIG. 2 shows a plurality of waveforms in accordance with some embodiments;

FIG. 3 shows an alternative waveform having the indicia in accordance with alternative embodiments;

FIG. 4 shows an alternative waveform having the indicia in accordance with alternative embodiments;

FIG. 5 shows an alternative embodiment of a sleep study device in accordance with some embodiments; and

FIG. 6 shows a method in accordance with at least some embodiments.

### Notation and Nomenclature

Certain terms are used throughout the following description and claims to refer to particular system components. This document does not intend to distinguish between components that differ in name but not function.

In the following discussion and in the claims, the terms “including” and “comprising” are used in an open-ended fashion, and thus should be interpreted to mean “including, but not limited to . . . ”. Also, the term “couple” or “couples” is intended to mean either an indirect or direct connection. Thus, if a first device couples to a second device, that connection may be through a direct connection, or through an indirect connection via other devices and connections.

Further, use of the terms “pressure,” “applying a pressure,” and the like shall be in reference herein, and in the claims, to gauge pressure rather than absolute pressure.

### DETAILED DESCRIPTION OF THE PREFERRED EMBODIMENTS

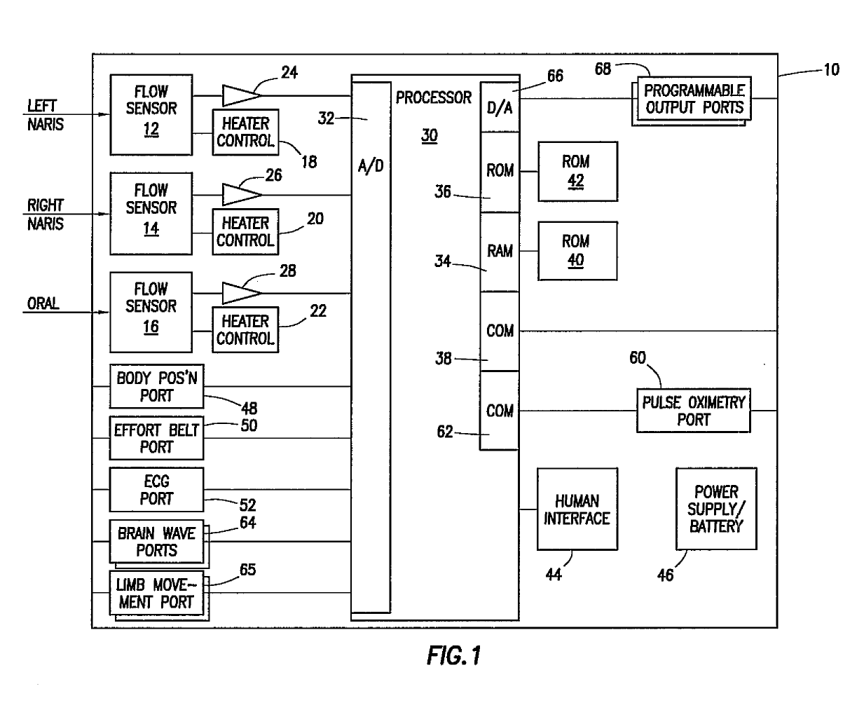

FIG. 1 illustrates, in block diagram form, a sleep study device 10 in accordance with at least some embodiments of the invention. The device 10 of FIG. 1 may be used as an interface between various patient sensors and the computer system of a sleep lab. The device 10 may comprise a flow sensor 12 that fluidly couples to a left naris of a patient, possibly by way of a first plenum of a dual lumen cannula (not specifically shown). The device 10 also comprises another flow sensor 14 that couples to a right naris of a patient, possibly by way of a second plenum of the dual lumen cannula. The device may also comprise a third flow sensor 16 which fluidly couples to the mouth of the patient. In accordance with at least some embodiments of the invention, the flow sensors 12, 14 and 16 are mass flow sensors available from Microswitch (a division of Honeywell Corp. of Morris Township, N.J.) having part number AWM92100V. However, other mass flow sensors and pressure sensors (such as a part no. MPXV5004DP pressure transducer from Motorola of Schaumburg, Ill.) may be used in place of the mass flow sensors. In embodiments using the mass flow sensors noted above, the device 10 may comprise heater control circuits 18, 20 and 22. Mass flow sensors of differing technology may not require heater control circuits.

The sleep study device 10 of FIG. 1 may also comprise amplifiers 24, 26 and 28 coupled to the flow sensors 12, 14 and 16 respectively. The purpose of amplifiers 24, 26 and 28 is to amplify the output signals propagating from each of the flow sensors. Depending on the type of flow sensors used, amplifiers 24, 26 and 28 may not be needed. Each flow sensor 12, 14 and 16 produces an output signal that has an attribute that changes proportional to the instantaneous airflow rate. Any attribute of an electrical signal may be used, such as frequency, phase, current flow, or possible a message based system where information may be coded in message packets. In some embodiments each sensor produces an output signal whose voltage is proportional instantaneous airflow rate.

The sleep study device 10 also comprises a processor 30, shown to have an on-board analog-to-digital (A/D) converter 32, on-board random access memory 34, on-board read-only memory (ROM) 36, as well as an on-board serial communication port 38. In embodiments where these devices are integral with the processor, the processor may be any of a number of commercially available microcontrollers. Thus, the processor 30 could be a microcontroller produced by Cypress Micro Systems having a part no. CY8C26643. In alternative embodiments, the functionality of the microcontroller may be implemented using individual components, such as an individual microprocessor, individual RAM, individual ROM, and an individual A/D converter. Random access memory, such as RAM 34, may provide a working area for the processor to temporarily store data, and from which programs may be executed. Read-only memory, such as ROM 36, may store programs, such as an operating system, to be executed on the processor 420. ROM may also store user-supplied programs to read respiratory data and to produce indicia indicative of the start of each inhalation and exhalation (discussed more below). Although microcontrollers may have on-board RAM and ROM, some embodiments may have additional RAM 40 and/or additional ROM 42 coupled to the processor 30. The RAM 40 may be the location to which the processor writes sleep data, and in some embodiments where the processor writes the indicia indicative of the start of each inhalation and exhalation. The RAM 40 may be selectively coupled and decoupled from the sleep study device, and sleep data may be transferred to other computers using RAM 40. The RAM 40 may be, for example, a secure digital interface memory card, such as a SDSDB or SDSDJ card produced by SanDisk of Sunnyvale, Calif. When using memory such as a secure digital interface memory card, a card reader may be used, such as a card reader part number 547940978 manufactured by Molex Incorporated of Lisle, Ill. Alternatively, the sleep data may be transferred to external devices by way of digital communications, such as through the communications port 3S.

In some embodiments, the sleep study device comprises a human interface 44 coupled to the processor 30. The human interface may comprise a data entry device, such as a full or partial keyboard, along with a display device, such as liquid crystal display. The sleep study device 10 may also comprise a power supply 46. In accordance with at least some embodiments of the invention, the power supply 46 is capable of taking alternating current (AC) power available at a wall outlet and converting it to one or more direct current (DC) voltages for use by the various electronics within the system. In alternative embodiments the sleep study device 10 is portable, and thus the power supply 46 has the capability of drawing current from on-board or external batteries, and converting to voltages needed by the devices within the sleep study device. In yet further embodiments, the power supply 46 may be external to the sleep study device 10.

Still referring to FIG. 1, a sleep study device 10 in accordance with embodiments of the invention may also couple to various other devices to aid in collecting data regarding the diagnosis of sleep disordered breathing. For example, in some embodiments the sleep study device 10 may have a body position port 48 coupled to the processor 30 by way of the A/D converter 32. The body position port 48 may couple to any commercially available body position indicator, such as a body position indicator having part no. 1664 produced by Pro-Tech Services, Inc. of Mukilteo, Wash. The processor, executing a program, writes body position data to the RAM 34, and/or RAM 42 for later analysis, or the processor may write a body position indication to one of the programmable output ports (discussed below).

Some embodiments may also comprise an effort belt port 50 electrically coupled to the processor 30 by way of the A/D converter 32. An effort belt, strapped around a patient's chest, measures increases and decreases in chest circumference as an indication of the patient's breathing effort. Thus, the effort belt port 50 may couple to any commercially available effort belt, such as an effort belt having part no. 1582 produced by Pro-Tech Services, Inc. In addition to (or in place of) the effort belt around the patient's chest, an effort belt may also be strapped around the patents abdomen. In cases where two efforts belts are used, an additional effort belt port (not specifically shown) is used. The processor, executing a program, writes effort data to the RAM 34 and/or RAM 40 for later analysis, or the processor may write an indication of effort to one of the programmable output ports (discussed below).

Some embodiments may also comprise an electrocardiograph (ECG) port 52 electrically coupled to the processor 30 by way of the A/D converter 32. An ECG analysis provides information on electrical potentials that occur during the patient's heart beat. Thus, the ECG port 52 may couple to any commercially available ECG device and/or sensors. The processor, executing a program, writes ECG data to the RAM 34 and/or RAM 40 for later analysis, or the processor may write an indication of measured ECG potentials to one of the programmable output ports (discussed below).

Some embodiments may also comprise a pulse oximetry port 60 electrically coupled to the processor 30 by way of the communication port 62. While FIG. 1 shows the pulse oximetry port 60 coupled to communication port 62, communication port 38 may serve a dual function, communicating with other computers and facilitating communication to an attached pulse oximetry device. A pulse oximeter provides information as to the patient's heart rate and blood oxygen saturation. Thus, the pulse oximetry port 60 may couple to any commercially available pulse oximeter device, such as a pulse oximeter part no. 4518-000 from Nonin Medical of Plymouth, Minn. The processor, executing a program, may write pulse and blood oxygen saturation data to the RAM 34 and/or RAM 40 for later analysis, or the processor may write an indication of heart rate and blood oxygen saturation to one of the programmable output ports (discussed below).

Some embodiments may also comprise brain wave ports 64 electrically coupled to the processor 30 by way of the A/D converter 32. Each brain wave port provides information on electrical potentials associated with a particular portion of the patient's brain. Thus, the brain wave ports 64 may couple to any commercially available electrodes. The processor, executing a program, writes brain wave data to the RAM 34 and/or RAM 40 for later analysis, or the processor may write an indication of brain wave data to one of the programmable output ports (discussed below).

Some embodiments may also comprise limb movement ports 65 electrically coupled to the processor 30 by way of the A/D converter 32. Each limb movement port provides information on movement of the patient's limbs (i.e., legs, arms). Thus, the limb movement ports 65 may couple to any commercially available limb movement detection devices, such as accelerometers. The processor, executing a program, writes limb movement data to the RAM 34 and/or RAM 40 for later analysis, or the processor may write an indication of limb movement data to one of the programmable output ports (discussed below).

When the sleep study device 10 is used in a dedicated sleep lab, the sleep study device 10 gathers data and provides the data (in various forms) to other equipment. For example, the sleep study device 10 may couple to and communicate with other equipment using packet-based messages by way of the communications port 38. By way of the communications port 38, the sleep study device 10 sends some or all the raw data, various values from the input ports (e.g., ports 48, 50, 52, 60 and 64), derived values such as scoring bar data (discussed below), and indicia indicative of the start of each inhalation and exhalation. In addition to, or in place of, the communications Through communications port 38, the sleep study device may drive analog data through various output signal ports coupled to the digital-to-analog (D/A) converter 66. For example, the processor 30 may calculate and drive analog output signals to the programmable output ports 68. The data provided from the device 10 to upstream devices, whether by way of packet-based messages or through the analog output ports 68, may be: left naris instantaneous airflow rate; right naris instantaneous airflow rate; the combined left naris and right instantaneous airflow rate; the difference between the instantaneous left and right naris airflow rate; the instantaneous oral airflow rate; combined instantaneous oral, left naris and right naris airflow rate; instantaneous oral airflow rate minus the combined left and right naris instantaneous airflow rate; combined instantaneous oral and left naris airflow rate; combined instantaneous oral and right naris airflow rate; instantaneous oral airflow rate minus the left naris instantaneous airflow rate; instantaneous oral airflow rate minus the right naris instantaneous airflow rate; snore signal of the left naris; snore signal of the right naris; snore signal detected at the mouth; combined left and/or right and/or oral snore signals; a derived scoring bar; or signals from limb movement sensors. Any of these signals may be useful to a polysomnographer in performing manual scoring of sleep data, or verifying automatic scoring.

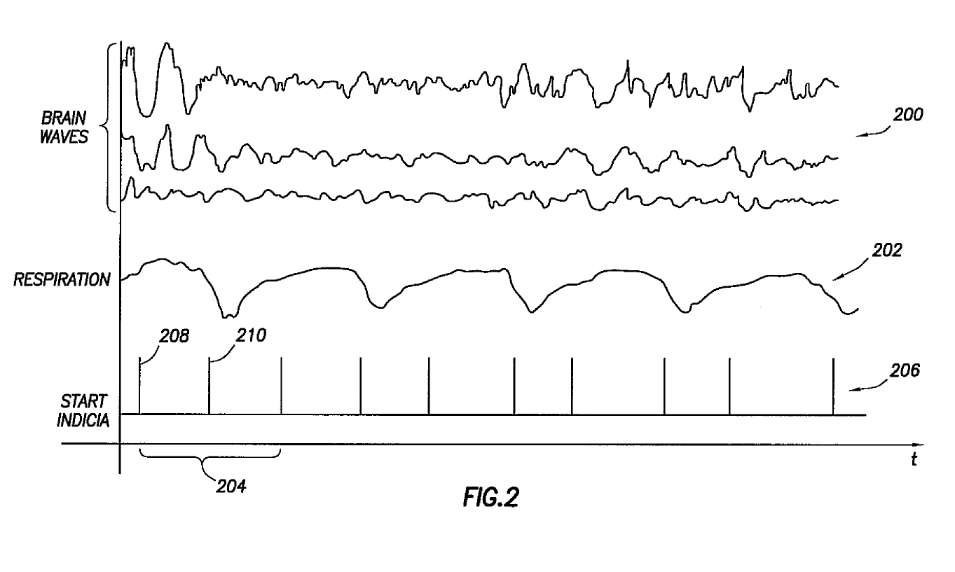

One of the difficulties in manually scoring sleep data by a polysomnographer is correlating in time various events, such as correlating fluctuation of a particular brain wave with the start of an inhalation of a respiration. In order to address these difficulties, the sleep study device 10, in addition to or in place of any of the signals noted in the preceding paragraph, also provides an indicia indicative of the start of an inhalation portion of a respiration, and may also provide an indicia indicative of the start of an exhalation portion of the respiration. FIG. 2 illustrates a plurality of plots of parameters of interest in a sleep study as a polysomnographer would see them on a display device. The display device creates the illustrative plots of FIG. 2 by: reproducing in video form time-varying analog signals provided by the sleep study device; reproducing in video form the signals conveyed by way of packet-based messages; or both.

In particular, FIG. 2 illustrates a plurality of plots of brain waves 200, a plot of a value indicative of respiration 202, and a plot illustrating the indicia indicative of start of inhalation and start of exhalation 204. Comparing just the plot of the respiration 202 against the brain waves 200, it is difficult if not impossible to ascertain the start of each inhalation, and the start of each exhalation, in relation to the brain waves 200. However, in accordance with embodiments of the invention indicia are provided, where each indicia is indicative of the start of an inhalation or the start of an exhalation. For example, in the complete respiration within time period 204, in the start indicia signal 206 a first voltage spike or impulse function 208 indicates the beginning of the inhalation portion of the respiration. Likewise, impulse function 210 indicates the beginning of the exhalation portion of the respiration. Each subsequent voltage spike or impulse function marks the start of an inhalation or the start of an exhalation.

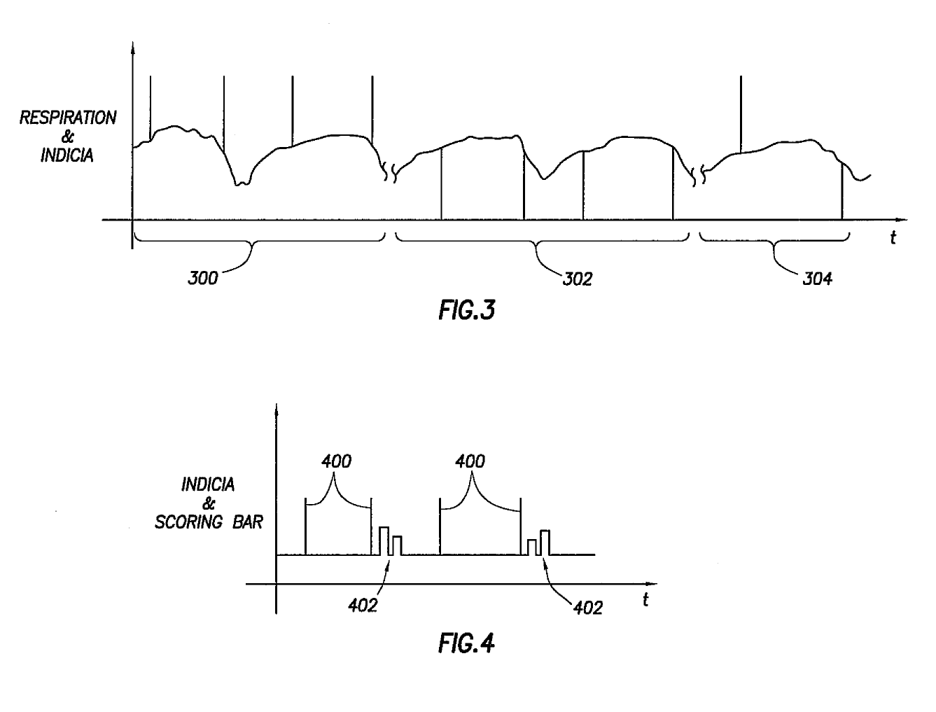

In the embodiments of the indicia indicative of start of an inhalation or exhalation illustrated in FIG. 2, the indicia are provided alone on a single horizontal plot; however, the indicia may be provided together with other information (e.g., a representation of the respiration). FIG. 3 illustrates several alternative embodiments. In the first region 300, the sleep study device 10 combines the positive going indicia with the signal representative of respiration. In region 302, the sleep study device 10 combines negative-going indicia with the signal representative of respiration. In region 304, the sleep study device 10 combines-positive going indicia for the start of inhalation, and negative-going indicia for the start of exhalation, with the signal representative of respiration. The combined signals of FIG. 3 could be analog signals produced by one of the programmable output ports 68 and reproduced in visual from on a display device, or the signals could be a stream of packet-based messages where the values the various points of the plot are contained in the messages and are reproduced on a display device.

At least some embodiments of the invention combine the indicia with a “scoring bar” output. A scoring bar is a waveform whose height (or width) is indicative of the volume of the last inhalation, exhalation, or both. Moreover, a running average bar may also provide the user a running average breath volume. For more information regarding scoring bars, running average bars and how to create them, the reader is directed to co-pending and commonly assigned patent application Ser. No. 11/226,570 titled “Method and system of scoring sleep disordered breathing,” which application is incorporated by reference herein as if reproduced in full below. FIG. 4 shows an illustrative output signal of the device 10 where the indicia and the scoring/running average bars are combined in a single output signal. In particular, FIG. 4 illustrates a plurality of indicia 400, and interspersed between selected indicia (e.g., just after each start of exhalation indicia), the scoring and running bars 402 may be placed. Here again, the combined signal of FIG. 4 could be an analog signal produced by one of the programmable output ports 68, or could be a stream of packet-based messages where the values the various points of the plot are contained in the messages and are reproduced on a display device.

The indicia discussed to this point have been voltage spikes or impulse functions; however, the indicia may take any suitable form. For example, a voltage spike indicative of the start of an inhalation may be taller (e.g., full scale deflection) than the spike indicative of the start of an exhalation (e.g., three-fourths full scale deflection). Further still, the indicia may be bars, similar to the bars 402, but of predetermined size and width. Further, the indicia may be predetermined shapes, one of whose features is indicative of the start of an inhalation or exhalation (e.g., leading edge of a triangular shape, trailing edge of a triangular shape). In cases where sleep study device provides signals to the upstream devices in the form of packet-based messages or streams of digital values, the indicia may be a predetermined value or series of values.

In the various embodiments discussed to this point, the sleep study device 10 is used as an interface between a patient and a computer system of a sleep lab. In alternative embodiments, the sleep study device 10 is used as an unattended sleep study device, with the device 10 storing relevant data, including the indicia of the start of inhalation and exhalation, to a memory, such as RAM 34 or RAM 40. The data may then be later transferred to other systems by way of a removable RAM 40, or by communication of the data over the communication port 38. Further still, in some embodiments the device 10 has human interface 44 that comprises a display screen, such as a liquid crystal display, and in these embodiments the device 10 may display various portions of the collected data, and also the indicia, on the display.

FIG. 5 illustrates alternative embodiments of the sleep study device. In particular, FIG. 5 illustrates a sleep study device 100 that comprises a base unit 102 and a portable unit 104. Much like the device 10, the device 100 is used an interface between various patient sensors and the computer system of a sleep lap; however, in this case the portable unit 104 may attached to and/or travel with the patient, thus freeing the patient from being constrained to a bed proximate to a point where the various wires and cannulas couple. This feature is particularly useful when, during a sleep study, the patient needs to urinate. The base unit 104 comprises a processor 106 that couples to the computer of sleep lab by way of one or more programmable analog output ports 108, or by way of digital communications over the communicate port 110 of the processor 106. Rather than coupling to sensors and ports within the same enclosure, as in FIG. 1, the processor 106 couples to the various sensors and ports by wirelessly communicating to the portable unit 104 through the wireless communication module 105.

Portable unit 104 comprises one or more sensors 112, which could be either the flow through sensors discussed above, or the pressure sensors discussed above. Also, portable unit comprises one or more ports 114, which could comprise: a body position sensor port; an effort belt sensor port; an ECG sensor port; a pulse oximeter sensor port; one or more brain wave ports; or one or more limb movement ports. The sensors and ports in the portable unit 104 are shown in shorthand notation so as not to unduly complicate the drawing. The portable unit 104 further comprises a processor 116 that couples to the various ports through an A/D converter 120 (or possibly a communication port in the case of the pulse oximeter sensor), and also couples to the base unit through a wireless communication module 120. Finally, the portable unit further comprises a battery 122. Processor 116 reads the various parameters of interest from the sensors 112 and the ports 114, and forwards the information to the base unit 102 wirelessly using the wireless communication module. The processor 106 of the base unit 106 receives the parameters, calculates data such as the indicia and the scoring bars, and provides the parameters and calculated data to upstream devices by way of the analog programmable output ports 108 and/or the communication port 110. In alternative embodiments, the processor 116 in the portable unit could perform some or all of the calculations; however, performing number intensive calculations requires additional power by the processor, thus reducing the amount of time the portable unit 104 could operating on a single battery charge or single set of batteries. In yet still other alternative embodiments, some of the electrical signals decoded from the wireless communication module could couple directly to the output ports 108, without the need of the signals to propagate through the processor 106.

The wireless communication between the portable unit 104 and the base unit 102 could take many forms, and thus the wireless communication modules too could take many forms. In some embodiments, the wireless communication is an optical coupling, such as by way of an infrared signal carrying the information. In other embodiments, the wireless communication is by way of electromagnetic waves. In these embodiments, the protocol of the communication could be any of a variety of currently existing, or after-developed, protocols (e.g., Bluetooth, EEE 802.11, dedicated point-to-point radio, or the lice).

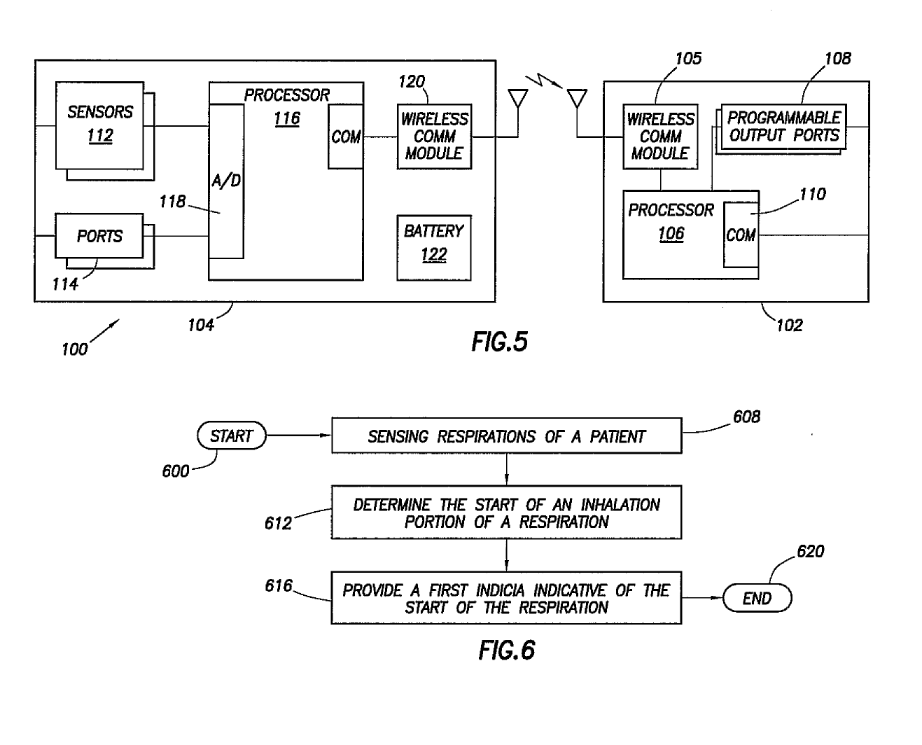

FIG. 6 illustrates a method in accordance with embodiments of the invention. In particular, the method starts (block 600) and proceeds to sensing respirations of a patient (block 608). Sensing the respiration may take many forms, such as sensing airflow associated with the respirations, or sensing a pressure proximate to a breathing orifice of the patient, the pressure indicative of the respirations. The sensors used may be a part of an interface device between the patient and computers of a sleep lab, with the sensors either integral to the interface device, or part of a portable unit wirelessly communicating to a base unit. Thereafter, a determination is made as the point in time of the start of an inhalation of a respiration (block 612). The determination may take any suitable form (e.g., determining the point in time a signal from a flow through sensor passes the zero flow point, or where a detected pressure passes the zero gauge pressure point). Of course, the determination is not limited to determining the start of an inhalation, and in other embodiments also comprises determining the start of a corresponding exhalation.

Still referring to FIG. 6, after the one or more determinations are made, an indicia is provided, the indicia indicative of the start of the inhalation portion. The indicia may take many forms. In systems where the indicia is provided by way of an analog output port, the indicia may be a voltage spike corresponding in time to when the inhalation starts, or some other predetermined voltage excursion (e.g., positive-going voltage spike, negative-going voltage spike, or other predetermined waveform shape). In systems where the indicia are provided by way of a stream of digital messages, each indicia may be a predetermined value or series of values. In systems where the indicia are stored in memory, each indicia may likewise be a predetermined value or series of values. In systems where the sensed parameters are displayed directly on a display device (possibly without being first produced in analog or digital form for communication), the indicia may be a visual indicator on a plot of information.

From the description provided herein, those skilled in the art are readily able to combine software created as described with appropriate general purpose or special purpose computer hardware to create a computer system and/or computer subcomponents embodying the invention, to create a computer system and/or computer subcomponents for carrying out the method of the invention, and/or to create a computer-readable medium for storing a software program to implement the method aspects of the invention.

The above discussion is meant to be illustrative of the principles and various embodiments of the present invention. Numerous variations and modifications will become apparent to those skilled in the art once the above disclosure is fully appreciated. For example, using the devices 10 or 100 with a nasal cannula only a portion of the total respiratory volume will be detected; however, the various techniques described work equally well even when only a portion of the total volume is detected. In alternative embodiments, a nasal mask, or a system comprising nasal pillows to seal to the nostrils, may be used such that substantially all the respiratory volume is measured, and this too falls within the contemplation of the invention. Thus, in this description and in the claims the terms “volume” and “total volume” may mean measured volume, whether that measured volume comprises some or all the respired volume. In the various embodiments described above, the signal processing to create the signals to drive to the programmable analog output ports 68, 108 is shown to be done by way of processor; however, this processing may alternatively be done with a trigger circuit of discrete components without departing from the scope and spirit of the invention. Moreover, while the various embodiments show the sensors coupled to the processor, and then the processor driving the analog output ports, for those sensors that inherently create analog signs the sensors may drive the analog output ports directly. It is intended that the following claims be interpreted to embrace all such variations and modifications.

What is claimed is:

1. A method comprising:
sensing respirations of a patient;
determining the start of an inhalation portion of a respiration; and
providing a first indicia indicative of the start of the inhalation portion, the first indicia associated with a representation of the respiration.

2. The method as defined in claim 1 wherein providing further comprises driving the first indicia in an analog output signal.

3. The method as defined in claim 2 wherein driving further comprises driving the first indicia and the representation of the respiration in the analog output signal.

4. The method as defined in claim 2 wherein driving the first indicia further comprises driving an impulse function in the analog output signal.

5. The method as defined in claim 1 wherein providing further comprises providing the first indicia in a data file storing data representative of the respiration.

6. The method as defined in claim 1 wherein providing further comprises providing the first indicia in a message stream forward to a computer system.

7. The method as defined in claim 1 wherein providing further comprises providing the first indicia as a visual indicator on a plot of the representation of the respiration on a display device.

8. The method as defined in claim 7 wherein providing further comprises displaying an impulse function.

9. The method as defined in claim 1 further comprising
determining the start of an exhalation portion of the respiration; and
providing a second indicia indicative of the start of the exhalation portion, the second indicia associated with a representation of the respiration.

10. The method as defined in claim 1 wherein sensing further comprises one selected from the group: sensing respiratory airflow through a flow through sensor; or sensing pressure associated with respiratory airflow.

11. A system comprising:
a sensor configured to sense respiratory airflow of a patient;
an electrical output port; and
a trigger circuit coupled to the sensor and the output port, the trigger circuit configured to sense a respiration of the patient using signals from the sensor;
wherein the trigger circuit is further configured to provide a first indicia indicative of a start of an inhalation portion of the respiration, the first indicia supplied to the electrical output port.

12. The system as defined in claim 11 wherein the trigger circuit further comprises a processor configured to sense a respiration of the patient using signals from the sensor and to provide a first indicia indicative of a start of an inhalation portion of the respiration.

13. The system as defined in claim 11 wherein the electrical output port is an analog output port, and wherein the first indicia is a voltage spike proximate in time to the start of the inhalation portion.

14. The system as defined in claim 11 wherein the electrical output port is a digital communication port, and wherein the first indicia is a value indicative of the start of the inhalation portion.

15. The system as defined in claim 11 wherein the trigger circuit is further configured to provide a second indicia indicative of a start of an exhalation portion of the respiration, and the second indicia supplied to the electrical output port.

16. The system as defined in claim 11 wherein the trigger circuit is further configured to generate a representation of the respiration, and to provide the representation to the electrical output port.

17. The system as defined in claim 11 wherein the sensor wirelessly couples to the trigger circuit.

18. The system as defined in claim 11 wherein the sensor Her comprises one selected from the group: a flow through sensor; a pressure sensor.

19. A computer readable medium storing instructions that, when executed by a processor, cause the processor to:
obtain data regarding respirations of a patient;
determine a start of an inhalation portion of a respiration;
provide a first indicia to a display device, the first indicia indicative of the start of the inhalation portion.

20. The computer-readable medium as defined in claim 19 wherein the instructions executed by the processor case the processor further to provide a second indicia to the display device, the second indicia indicative of the start of an exhalation portion of the respiration.

21. The computer-readable medium as defined in claim 19 wherein when the processor provides the instructions cause the processor to provide the first indicia along with a representation of the respiration.
